(19) United States
(12) Patent Application Publication
(10) Pub. No.: US 2026/0240872 A1
(43) Pub. Date: Aug. 20, 2026

(54) COMBINATION OF METFORMIN AND GLIBENCLAMIDE IN THE TREATMENT OF PARKINSON'S DISEASE

(71) Applicant: CXS THERAPEUTICS, FONDETTES (FR)

(72) Inventor: Guillaume BRACHET, FONDETTES (FR)

(73) Assignee: CXS THERAPEUTICS, FONDETTES (FR)

(21) Appl. No.: 19/160,753

(22) PCT Filed: Mar. 7, 2024
(86) PCT No.: PCT/EP2024/056052

§ 371 Date: Aug. 28, 2025

(30) Foreign Application Priority Data
Mar. 7, 2023 (EP) 23160619.5
Nov. 21, 2023 (EP) 23315435.0

(51) Int. Cl. A61K 31/64; A61K 31/155; A61K 45/06; A61P 25/16

(52) U.S. Cl. CPC A61K 31/64; A61K 31/155; A61K 45/06; A61P 25/16

(57) ABSTRACT

The present invention provides a pharmaceutical composition comprising metformin and glibenclamide for use in the treatment of Parkinson's disease. The invention also comprises a combined administration of metformin and glibenclamide. In a preferred embodiment, the administration is made through oral route.

The present invention provides a pharmaceutical composition comprising metformin and glibenclamide for use in the treatment of Parkinson's disease. The invention also comprises a combined administration of metformin and glibenclamide. In a preferred embodiment, the administration is made through oral route.

### TECHNICAL FIELD

The technical domain of the invention is the treatment of Parkinson's disease.

### BACKGROUND ART

Parkinson's disease (PD) has a considerable impact on society, with around 6.1 million people affected worldwide in 2016. For reasons that are not yet fully understood, the incidence and prevalence of this disease have increased rapidly over the past two decades. The personal consequences of Parkinson's disease are enormous, with the duration of the disease potentially spanning decades, with accumulating disability for those affected. Parkinson's disease also has profound consequences for caregivers, most of whom are under excessive pressure.

Parkinson's disease is a complex multifactorial neurodegenerative disease concerning mainly elderly patients. Dopaminergic neurons from the substantia nigra undergo progressive decline, due to an accumulation of modified proteins, which aggregate and form Lewy bodies. Protein aggregation and accumulation inside these specific cells is unexplained, however the progressive loss of dopaminergic neurons leads to rigidity, akinesia and tremor, a syndrome triad linked with Parkinson's disease and syndromes (Bloem, Okun, et Klein 2021). Some genetic polymorphisms are known to be risk factors, but are not systematically found, and some associations with physical trauma or prolonged exposure to certain toxic substances have been identified.

Protein aggregation follows cellular stress involving reactive oxygen species, mitochondrial metabolism and more recently, glucose metabolism has been pointed out as a source of reactive oxygen species which react with intracellular proteins, and cause accumulation of Lewy bodies. Glycolysis produces reactive metabolites such as 1,3-bisphosphoglycerate which are sources of reactive oxygen species (Heremans et al. 2022a). The specific sensitivity of motoneurons from the substantia nigra remains however unclear (Heremans et al. 2022b).

Up to date, there are no medicines able to slow down the progression of the disease, i.e. the neurodegeneration of dopaminergic neurons inside the substantia nigra, which can either be fast or slow, evolving over decades or months before and after the onset of symptoms. The authorized drugs either act by increasing dopamine release by the remaining neurons, or by acting as dopamine substitutes. Combined with physical activity, they help patients retaining autonomy, but do not slow the neurodegeneration (Radhakrishnan et Goyal 2018).

Glucose metabolism is pointed out as a potential target for disease-modifying treatments. Indeed, Mediterranean diet, which consists in a variety of crude fruits, vegetables, vegetal oils, fish and in general few fast-acting carbohydrates, has been linked with a mild preservation of motor functions in patients with Parkinson's disease (Paknahad et al. 2020). This diet is associated to a metabolic switch towards ketosis.

The finding that a metabolic switch from glycolysis to ketosis is supported by the efficacy of metformin, an anti-hyperglycemic antidiabetic drug, in preclinical models of Parkinson's disease and in protection non-dementia vascular cognitive impairment and abnormal glucose metabolism (Mor et al. 2020b). Yet, only mild improvements or even worsening of the disease have been observed so far in Parkinson's disease (Mohamed Alrouji et al. 2023).

Metformin has a triple effect in neuroprotection. First, by lowering glucose levels, it decreases the availability of serum glucose, which is transformed into cyclic 1,3-bisphosphoglycerate during glycolysis. Second, it forces glycerol excretion, thus detoxicating the intermediary metabolites responsible for the accumulation of cyclic 1,3-bisphosphoglycerate. Finally, by promoting neurogenesis (Mor et al. 2020b; Markowicz-Piasecka et al. 2017; Wang et al. 2012).

Metformin could therefore be of interest for the treatment of Parkinson's disease. The major drawback concerning this molecule lies in the tolerance issues. Metformin is mainly cleared through kidney ultrafiltration. In case of insufficient kidney function, it accumulates and causes a life-threatening increase in serum lactate, resulting in lactic acidosis. Thus, the molecule is contraindicated for use in patients above 65 years of age when renal function ascertained by blood clearance drops below 30 mL/min.

However, patients concerned by Parkinson's disease are aged 75+ for half, and have a tendency towards cachexia, which makes them at risk for metformin use (Barichella, Cereda, et Pezzoli 2009).

Based on the hypothesis that neuroprotection comes from preventing cyclic 1,3-bisphosphoglycerate accumulation inside dopaminergic neurons from the substantia nigra, the ideal treatment would have the properties of metformin without the side effects endangering the target population.

Taking into consideration the persistent need for medication to slow down the progression of Parkinson's disease, the applicant identified a synergistic and original combination of molecules which surprisingly combine efficacy and low side-effects, thanks to a specific dosage of metformin and glibenclamide, allowing for the safe chronic treatment of a vast majority of patients. Whilst each compound alone has no effect or can even be toxic to dopaminergic neurons, low combinational dosing increases cell fitness characterized by neurite length and prevents neurodegeneration.

### SUMMARY

The present invention relates to a pharmaceutical composition comprising metformin and glibenclamide for use in the treatment of Parkinson's disease.

Said pharmaceutical composition comprises a therapeutically effective amount of metformin and glibenclamide.

In an embodiment, said metformin is present in an amount providing an effective dose of between 0.025 mg/day and 450 mg/day.

In an embodiment, said metformin is present in an amount providing an effective dose of between 0.025 mg/day and 250 mg/day.

Preferably, said metformin is present in an amount providing an effective dose of 0.025 mg/day, or 0.050 mg/day, or 0.075 mg/day, or 0.1 mg/day or 0.125 mg/day, or 0.150 mg/day, or 0.175 mg/day, or 0.200 mg/day, or 0.225 mg/day, or 0.250 mg/day, or 0.275 mg/day, or 0.300 mg/day, or 0.325 mg/day, or 0.350 mg/day, or 0.375 mg/day, or 0.400 mg/day, or 0.425 mg/day, or 0.450 mg/day, or 0.475 mg/day, or 0.500 mg/day, or 0.525 mg/day, or 0.550 mg/day, or 0.575 mg/day, or 0.600 mg/day, or 0.625 mg/day, or 0.650 mg/day, or 0.675 mg/day, or 0.700 mg/day, or 0.725 mg/day, or 0.750 mg/day, or 0.775 mg/day, or 0.800 mg/day, or 0.825 mg/day, or 0.850 mg/day, or 0.875 mg/day, or 0.900 mg/day, or 0.925 mg/day, or 0.950 mg/day, or 0.975 mg/day, or 1 mg/day, or 1.25 mg/day, or 1.50 mg/day, or 1.75 mg/day, or 2 mg/day, or 2.25 mg/day, or 2.50 mg/day, or 2.75 mg/day, or 3 mg/day, or 3.25 mg/day, or 3.50 mg/day, or 3.75 mg/day, or 4 mg/day, or 4.25 mg/day, or 4.50 mg/day, or 4.75 mg/day, or 5 mg/day, or 6 mg/day, 7 mg/day, 8 mg/day, 9 mg/day, or 10 mg/day, or 12.5 mg/day, or 15 mg/day, or 17.5 mg/day, or 20 mg/day, 22.5 mg/day, or 25 mg/day, 27.5 mg/day or 30 mg/day, or 32.5 mg/day, or 35 mg/day, or 37.5 mg/day, or 40 mg/day, or 42.5 mg/day, or 45 mg/day, or 47.5 mg/day, or 50 mg/day, or 60 mg/day, or 70 mg/day, or 80 mg/day, or 90 mg/day or 100 mg/day, or 125 mg/day, or 150 mg/day, or 175 mg/day, or 200 mg/day, or 225 mg/day, or 250 mg/day, or 275 mg/day, or 300 mg/day, or 325 mg/day, or 350 mg/day, or 375 mg/day, or 400 mg/day, or 425 mg/day, or 450 mg/day.

More preferably, said metformin is present in an amount providing an effective dose of about 50 mg/day.

In an embodiment, said glibenclamide is present in an amount providing an effective dose of between 0.001 mg/day and 2 mg/day.

In an embodiment, said glibenclamide is present in an amount providing an effective dose of between 0.001 mg/day and 1 mg/day.

Preferably, said glibenclamide is present in an amount providing an effective dose of 0.001 mg/day, or 0.005 mg/day, or 0.010 mg/day, or 0.015 mg/day, or 0.020 mg/day, or 0.025 mg/day, or 0.030 mg/day, or 0.035 mg/day, or 0.030 mg/day, or 0.035 mg/day, or 0.040 mg/day, or 0.045 mg/day, or 0.050 mg/day, or 0.055 mg/day, or 0.060 mg/day, or 0.065 mg/day, or 0.070 mg/day, or 0.075 mg/day, or 0.080 mg/day, or 0.085 mg/day, or 0.090 mg/day, or 0.095 mg/day, or 0.1 mg/day, or 0.15 mg/day, or 0.2 mg/day, or 0.25 mg/day, or 0.3 mg/day, or 0.35 mg/day, or 0.4 mg/day, or 0.45 mg/day, 0.5 mg/day, or 0.55 mg/day, or 0.6 mg/day, or 0.65 mg/day, or 0.7 mg/day, or 0.75 mg/day, or 0.8 mg/day, or 0.85 mg/day, or 0.9 mg/day, or 0.95 mg/day, or 1 mg/day, or 1.05 mg/day or 1.1 mg/day, or 1.15 mg/day, or 1.2 mg/day, or 1.25 mg/day, or 1.3 mg/day, or 1.35 mg/day, or 1.4 mg/day, or 1.45 mg/day, 1.5 mg/day, or 1.55 mg/day, or 1.6 mg/day, or 1.65 mg/day, or 1.7 mg/day, or 1.75 mg/day, or 1.8 mg/day, or 1.85 mg/day, or 1.9 mg/day, or 1.95 mg/day, or 2 mg/day.

In an embodiment, said glibenclamide is present in an amount providing an effective dose of 0.5 mg/day.

In an embodiment, the pharmaceutical composition according to the invention further comprises at least one other acceptable active pharmaceutical ingredient, and/or at least one acceptable pharmaceutical excipient or carrier.

In a preferred embodiment, said active pharmaceutical ingredient is another anti-Parkinson's disease agent.

### Definition

“Pharmaceutical composition” or “pharmaceutical formulation” refers to the formation of a pharmaceutical product in which different chemical substances, including the active drugs, are combined to produce a final medicinal product.

“Combination” in the present invention refers to the association of two compounds, e.g., metformin and glibenclamide, to be administered to the same subject. According to the present invention, said two compounds can be administered concomitantly or sequentially. Thus, according to the present invention, the administration of the first compound does not necessarily overlap with the administration of the second compound.

“Concomitantly” refers to an administration of metformin and glibenclamide at the same time, the administration of the first compound overlapping with the administration of the second compound.

“Sequentially” refers to an administration of metformin and glibenclamide which is not done at the same time. Glibenclamide or metformin is administered before the other molecule, and the administration of the first compound does not overlap with the administration of the second compound.

“Subject”, “individual” or “patient” refers to a mammal, preferably a human. In one embodiment, the subject is diagnosed with Parkinson's disease. In one embodiment, the subject is a patient, preferably a human patient, who/which is awaiting the receipt of, or is receiving, medical care or was/is/will be the subject of a medical procedure or is monitored for the development or progression of Parkinson's disease. In one embodiment, the subject is a male. In another embodiment, the subject is a female. In one embodiment, the subject is an adult. In another embodiment, the subject is a child.

“Therapeutically effective amount” or “therapeutically effective dose” refer to the amount or dose of metformin, to the amount or dose of glibenclamide, or to the amount or dose of both, in a combination according to the invention, that is aimed at, without causing significant negative or adverse side effects to the subject, slowing down or stopping the progression, aggravation, or deterioration of Parkinson's disease affecting the subject.

“Treating”, “treat” or “treatment” refers to therapeutic treatment, to prophylactic or preventative measures, or to both, wherein the object is to prevent or slow down Parkinson's disease. A subject is successfully “treated” if, after receiving a therapeutic amount of metformin and of glibenclamide in combination according to the present invention, the subject shows observable and/or measurable reduction in the consequences of Parkinson's disease, acknowledged by symptom reduction, restoration of previously lost or hindered symptomatic treatment efficacy.

“About” preceding a figure encompasses plus or minus 10%, or less, of the value of said figure. It is to be understood that the value to which the term “about” refers is itself also specifically disclosed.

“Idiopathic Parkinson's Disease” is a multisystemic synucleinopathy of the human nervous system with functional consequences and a diagnostic potential that extend beyond the nigrostriatal system. Intracerebrally, only a few predisposed types of nerve cells develop the inclusion body pathology that appears in the form of Lewy neurites, Lewy bodies, and Lewy plaques. Melanoneurons and other projection cells generating long axons that are unmyelinated or sparsely myelinated are particularly susceptible. This selective vulnerability on the part of specific neuronal populations as well as anatomically and functionally interconnected brain regions results in a distinctive topographic distribution pattern of brain lesions that is nearly consistent across autopsy cases and, as such, makes it possible to predict the intracerebral progress of IPD and stage it neuropathologically. In stage 1, the lesions are confined to predisposed induction sites: the brain stem dorsal visceromotor nucleus of the vagal nerve, intermediate reticular zone, and/or telencephalic bulbus olfactorius. In stage 2 cases, inclusion bodies begin to appear in portions of the caudal raphe nuclei (above all, the nucleus raphes magnus and obscurus), the gigantocellular reticular nucleus, and the coeruleus/subcoeruleus complex. The severity of the lesions in stage 1 typically increases in stage 2 cases, and the pathology at brain stem sites not only worsens throughout the following stages but is directed essentially upwards in the direction of the cerebral cortex. Neuronal damage begins in the mesencephalic substantia nigra, pars compacta in stage 3 and is accompanied by alterations in the tegmental pedunculopontine nucleus and in the prosencephalon (central subnucleus and basolateral complex of the amygdala, magnocellular nuclei of the basal forebrain, hypothalamic tuberomammillary nucleus). In stage 4, the disease process reaches the cerebral cortex (anteromedial temporal mesocortex) for the first time. During stage 4 and subsequent stages, IPD progresses into additional telencephalic regions, including chiefly the transentorhinal region, hippocampal formation, anterior cingulate mesocortex (all limbic loop structures), and insular and subgenual areas of the mesocortex (cortical components of the autonomic loop). The pathology that accrues in these and other nonsomatomotor system structures almost certainly leads to detectable olfactory impairment, deficits in responses to emotional stimuli, dysfunctions of visceromotor and endocrinal systems, and most probably diminished cognitive faculties, including, in some individuals, dementia. The reduced influence of input from limbic system high-order centers upon both the cerebral cortex and the brain stem reticular formation probably contributes to the affect-related deficits of the voluntary and emotional motor systems that typically become manifest in the course of IPD. In the final stages 5 and 6, the pathology advances until it occupies extensive stretches of the neocortex, beginning with the high-order sensory association and prefrontal areas, then the first order sensory and premotor fields, and eventually the primary sensory and primary motor fields of the mature neocortex. (Kelly del Tredici and Heiko Braak, Madame Curie Bioscience Database)

“Early onset Parkinson's disease” or “young onset Parkinson's disease (YOPD)” refers to a type of Parkinson's disease where the diagnosis is made for someone who is 21-50 years old. While common symptoms of Parkinson's may be similar no matter what age you are, the progression is often different: Young people often have more involuntary movement problems due to the most commonly prescribed Parkinson's disease medication, levodopa. Other problems associated with Parkinson's such as memory loss, confusion, and balance difficulties tend to be less frequent in young people with the disease.

“Parkinson's disease associated with aberrant splicing” refers to Parkinson's disease including aberrant splicing. At least PARK2, SNCAIP, LRRK2, SNCA, SRRM2, and MAPT are involved in aberrant AS events in PD patients (FU, Ru-Huei, et al. 2013).

PARK2 mutations are accompanied by an imbalance in programmed cell death systems in which non-apoptotic molecular mechanisms play the leading role (Konovalova et al. 2015). The PARK2 gene is responsible for almost half of cases of autosomal recessive Parkinson's disease with early onset (Olga Corti 2014).

Mutations within the MAPT gene encoding the microtubule-associated protein tau result in the clinical phenotype of frontotemporal dementia with parkinsonism. Genome-wide association studies have implicated MAPT H1 as a significant risk factor for Parkinson's disease (PD); however preliminary sub-haplotype analyses suggest that different genetic variants on the MAPT H1 haplotype associate with each of these parkinsonian disorders. (Ross Owen 2012).

Mutations in the leucine-rich repeat kinase 2 (LRRK2) gene are a common genetic causes of Parkinson's disease (PD), and also one of the strongest genetic risk factors in sporadic PD. The LRRK2 protein contains a GTPase domain and a kinase domain, and several protein-protein interaction domains (Yulan Xiong et al. 2017).

Synphilin-1 (SNCAIP) is a presynaptic protein that associates with synaptic vesicles (Ribeiro et al. 2002). It is associated with Parkinson's Disease (PD) because it is an intrinsic component of Lewy bodies (Wakabayashi et al. 2000) and a mutation of the SNCAIP gene has been identified in some PD patients (Marx et al. 2003), suggesting that accumulation of SNCAIP and its interaction with SNCA may be relevant for Lewy body formation in PD. SNCAIP is ubiquitinated by several different E3 ubiquitin-ligases, including Parkin (PARK2)

The alpha-synuclein gene (SNCA) was indisputably considered as the first pathogenic gene responsible for autosomal dominant PD, supported by the fact that its protein aggregation is thought to be the primary pathological hallmark of the patients, though only a few mutations were identified (Polymeropoulos et al., 1997; Fields et al., 2019).

SRRM2 is a RNA splicing factor which has been reported as being consistently dysregulated in different PD neuronal sources (Shehadeh et al. 2010).

“Trauma-induced Parkinson's disease” refers to Parkinson's disease linked to traumatic brain injury. Traumatic brain injury (TBI) has been implicated as a risk factor for PD. This includes mild TBI (mTBI), which is known to be responsible for a 56% higher risk of developing PD in U.S. Veterans, the risk increasing with severity of injury (Delic et al. 2020).

“Vascular Parkinson's disease” refers to Parkinson's disease related to vascular or pseudovascular injuries. This denomination includes progressive ambulatory impairment and abnormal white matter (WM) signal visible on neuroimaging.

“Drug-induced Parkinson's disease” refers to Parkinson's disease which followed the take of a drug. This subtype is often reversible after withdrawal of the causative drug. All known causative drugs were prescribed in non-neurological departments and over one half were prescribed in non-psychiatric departments; most were prescribed to treat depression or abdominal discomfort (Shiraiwa et al. 2018).

“Parkinson's disease in the elderly adult” refers to Parkinson's disease on patients exceeding the age of 65.

“Acceptable pharmaceutical excipient or carrier” refers to an excipient or carrier that does not produce an adverse, allergic or other untoward reaction when administered to a mammal, preferably a human. It includes any and all solvents, dispersion media, coatings, antibacterial and antifungal agents, isotonic and absorption delaying agents and the like. A pharmaceutically acceptable carrier or excipient refers to a non-toxic solid, semi-solid or liquid filler, diluent, encapsulating material or formulation auxiliary of any type. For human administration, preparations should meet sterility, pyrogenicity, general safety and purity standards as required by the regulatory offices such as the FDA or EMA.

“Anti-Parkinson's disease agent” refers to a molecule known to prevent, treat or slow down Parkinson's disease and/or its consequences.

“Administration” refers to the take of a drug, by whatever routes, in order to get through the target site in the patient's body.

“Oral administration” refers to a route of administration whereby pharmaceutical compositions are taken through the mouth, swallowed, and then processed via the digestive system.

“Rectal administration” refers to a route of administration using the rectum. Pharmaceutical compositions are absorbed by the rectum's blood vessels and flow into body's circulatory system, distributing the drug to the target site.

“Intramuscular administration” refers to the injection of a substance into a muscle. Muscles have larger and more numerous blood vessels than subcutaneous tissue, leading to faster absorption than subcutaneous or intradermal injections.

“Intravenous administration” refers to an administration directly into a person's veins.

“Subcutaneous administration” refers to the insertion of medications beneath the skin either by injection or infusion.

### DESCRIPTION OF EMBODIMENTS

The present invention thus relates to a pharmaceutical composition comprising metformin and glibenclamide for use in the treatment of Parkinson's disease.

Parkinson's disease is a brain disorder that causes unintended or uncontrollable movements, such as shaking, stiffness, and difficulty with balance and coordination. The pathophysiology of Parkinson's disease seems to result from the complex interplay of aberrant α-synuclein aggregation, dysfunction of mitochondria, lysosomes or vesicle transport, synaptic transport problems and neuroinflammation.

These pathological mechanisms lead to accelerated death of mainly dopaminergic neurons, but the neuropathology involves many other motor and non-motor circuits.

Loss of nigrostriatal dopaminergic cells causes a gradient of striatal dopamine depletion producing an imbalance between direct (facilitatory) and indirect (inhibitory) pathways through the basal ganglia, resulting in bradykinesia.

Neurophysiological recordings conceptualized bradykinesia as an imbalance between different oscillatory rhythms: too much beta activity (antikinetic) and too little gamma activity (prokinetic). More precisely, beta oscillations are associated with the deactivated dopaminergic state and disappear with dopaminergic drugs or deep brain stimulation.

A relatively new finding is that these pathological changes are accompanied by compensatory alterations in brain activity in areas initially unaffected by Parkinson's pathology, such as a shift towards more anterior cortico-striatal circuits and recruitment of cortical regions less connected to the basal ganglia (Bastiaan R Bloem et al., Parkison's disease, Lancet 2021.).

Metformin is the International Nonproprietary Name (INN) to designate the substance 1,1-dimethylbiguanide (CAS 657-24-9). Metformin is a largely use medication for the treatment of type 2 diabetes, especially used in the form of its hydrochloride C₄H₁₁N⁵·HCl. Classically, its role is to decrease the insulin resistance of the carbohydrate intolerant organism and to decrease the hepatic neoglucogenesis. Metformin can be prepared by known methods, or may be obtained from commercial sources (For example GLUCOPHAGE®, STAGID®, GLUMETZAR AND FORTAMET®). Metformin has the structure depicted below:

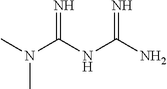

As used herein, the term “metformin” encompasses any prodrugs, pharmaceutically acceptable salts, hydrates and solvates thereof. In particular, the term “metformin” encompasses chlorhydrate and embonate salts, as well as the hydrochloride salts and mono-hydrochloride salt thereof, e.g., metformin hydrochloride and metformin mono-hydrochloride. The term “metformin” also encompasses the crystalline forms of said compound.

Glibenclamide, also known as glyburide, is an antidiabetic drug in a class of medications known as sulfonylureas, closely related to sulfa drugs. This molecule has a hypoglycemic effect, exhibit a good tolerance profile, is compatible for combined formulation, and exhibit synergy to allow for low dosing because of frequent polymedication in the target population. Glibenclamide can be prepared by known methods, or may be obtained from commercial sources (For example DAONIL®.) Glibenclamide has the structure depicted below:

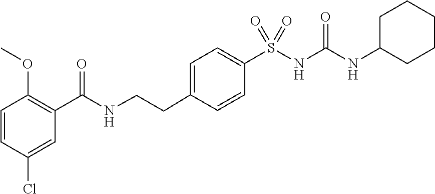 [SMILES: COc1ccc(Cl)cc1C(=O)NCCc1ccc(S(=O)(=O)NC(=O)NC2CCCCC2)cc1]

The Applicant surprisingly showed that the combined administration of metformin and glibenclamide significantly and synergistically result in neuroprotection in the context of Parkinson's disease by regulating the integrity of the BBB, and in situ by promoting the survival of neurons.

Moreover, having a low dose of metformin allows to prevent lactic acidosis and B12 deprivation, two major side effects of this molecule.

This combination, in the case of Parkinson's patient, and especially in elderly parkinsonian patients, is an advantage, limiting the metformin-related toxicity but retaining its intrinsic neuroprotective, antioxidant effects.

This combination of molecules works through a novel mechanism of action characterized by the Applicant.

A growing number of studies showed a strong link between metformin and the regulation of AMPK, mainly mediated by an inhibition, at the mitochondrial level, of the respiratory chain Complex I, as well as of Glycerol-3-Phosphate Dehydrogenase (Francesco Agostini et al., Int. J. Mol. Sci., 2022 and Vial et al., Front. Endocrinol., 7 May 2019). One of its main transporters, OCT1, is expressed on the surface of a large number of cell types, including neurons and endothelial cells. These mechanisms applied to the treatment of Parkinson's disease, should therefore be studied with the greatest attention.

Then, metformin is likely to exert a neuroprotective role mainly by regulating mitochondrial function:
By preventing the accumulation of toxic protein aggregates via the inhibition of Glycerol-3-Phosphate Dehydrogenase activity, a decrease in gluconeogenesis, and ultimately abnormal protein glycation events.
By preventing protein misfolding, oxidative stress and protein synthesis, as well as promoting the process of autophagy (regulation of mitochondrial AMPK levels).

The NLRP3 inflammasome complex has been associated with Blood-brain barrier (BBB) permeability and neuroinflammation (Lawrence et al. 2022). NLRP3-inflammasome is an intracellular protein complex and a key mediator of inflammation in many pathologies. Its activation by various effectors leads to the release of the pro-inflammatory cytokines IL-1B and IL-18, as well as the process of pyroptosis, a type of programmed cell death characterized by permeabilization of the plasma membrane by family members gasdermin proteins (Rebecca C. Coll et al., Trends in Pharmacological Sciences. 2022). A recent study showed that the use of glibenclamide is capable of improving the integrity of the BBB by reducing the activity of NLRP3 (mechanism explained by a reduction in cellular potassium efflux) and pyroptosis at the level endothelial cells (Fulin Xu et al., Brain Behav. 2019).

Then, Glibenclamide mainly mediates its neuroprotective effects by inhibiting the activity of the NLRP3 inflammasome and ultimately programmed cell death by pyroptosis. Two different places of action could constitute the targets of this mechanism:
Within the BBB, where it would help maintain the integrity of the barrier.
In-situ in the neuron, preventing the decrease in the cell population.

The concomitant use of the 2 molecules (metformin and glibenclamide) and the targeting of the previously mentioned mechanisms would result in neuroprotection in the context of Parkinson's disease by regulating the integrity of the BBB, and in situ by promoting the survival of neurons.

The present invention thus relates to a combination comprising metformin as described hereinabove and glibenclamide as described hereinabove for use in the treatment of Parkinson's disease.

A second object of the invention concerns a method of treating Parkinson's disease comprising the administration of metformin and glibenclamide.

According to a third object, the invention concerns the combined administration of metformin and glibenclamide, for use in the treatment of Parkinson's disease.

Another object of the present invention is a kit-of-parts comprising a first part comprising metformin and a second part comprising glibenclamide for use in the treatment of Parkinson's disease.

Another object of the present invention is the use of a combination comprising or consisting of metformin and glibenclamide as described hereinabove for the manufacture of a medicament for treating Parkinson's disease.

Another object of the present invention is a pharmaceutical combination comprising or consisting of metformin and glibenclamide as described hereinabove, and at least one pharmaceutically acceptable excipient, for use in the treatment of Parkinson's disease.

Another object of the invention is a medicament comprising or consisting of a combination of metformin and glibenclamide as described hereinabove, or a pharmaceutical combination as described hereinabove, or a kit-of parts as described hereinabove, for use in the treatment of a Parkinson's disease.

In an embodiment, the Parkinson's disease is early onset Parkinson's disease, idiopathic Parkinson's Disease, Parkinson's disease associated with aberrant splicing, trauma-induced Parkinson's disease, vascular Parkinson's disease, drug-induced or Parkinson's disease in the elderly adult.

It will be understood that the total daily usage of metformin and the total daily usage of glibenclamide in combination according to the invention will be decided by the attending physician within the scope of sound medical judgment.

Said pharmaceutical composition comprises a therapeutically effective amount of metformin and glibenclamide.

In an embodiment, said metformin is present in an amount providing an effective dose of between 0.025 mg/day and 450 mg/day.

In an embodiment, said metformin is present in an amount providing an effective dose of between 0.025 mg/day and 250 mg/day.

Preferably, said metformin is present in an amount providing an effective dose of 0.025 mg/day, or 0.050 mg/day, or 0.075 mg/day, or 0.1 mg/day or 0.125 mg/day, or 0.150 mg/day, or 0.175 mg/day, or 0.200 mg/day, or 0.225 mg/day, or 0.250 mg/day, or 0.275 mg/day, or 0.300 mg/day, or 0.325 mg/day, or 0.350 mg/day, or 0.375 mg/day, or 0.400 mg/day, or 0.425 mg/day, or 0.450 mg/day, or 0.475 mg/day, or 0.500 mg/day, or 0.525 mg/day, or 0.550 mg/day, or 0.575 mg/day, or 0.600 mg/day, or 0.625 mg/day, or 0.650 mg/day, or 0.675 mg/day, or 0.700 mg/day, or 0.725 mg/day, or 0.750 mg/day, or 0.775 mg/day, or 0.800 mg/day, or 0.825 mg/day, or 0.850 mg/day, or 0.875 mg/day, or 0.900 mg/day, or 0.925 mg/day, or 0.950 mg/day, or 0.975 mg/day, or 1 mg/day, or 1.25 mg/day, or 1.50 mg/day, or 1.75 mg/day, or 2 mg/day, or 2.25 mg/day, or 2.50 mg/day, or 2.75 mg/day, or 3 mg/day, or 3.25 mg/day, or 3.50 mg/day, or 3.75 mg/day, or 4 mg/day, or 4.25 mg/day, or 4.50 mg/day, or 4.75 mg/day, or 5 mg/day, or 6 mg/day, 7 mg/day, 8 mg/day, 9 mg/day, or 10 mg/day, or 12.5 mg/day, or 15 mg/day, or 17.5 mg/day, or 20 mg/day, 22.5 mg/day, or 25 mg/day, 27.5 mg/day or 30 mg/day, or 32.5 mg/day, or 35 mg/day, or 37.5 mg/day, or 40 mg/day, or 42.5 mg/day, or 45 mg/day, or 47.5 mg/day, or 50 mg/day, or 60 mg/day, or 70 mg/day, or 80 mg/day, or 90 mg/day or 100 mg/day, or 125 mg/day, or 150 mg/day, or 175 mg/day, or 200 mg/day, or 225 mg/day, or 250 mg/day, or 275 mg/day, or 300 mg/day, or 325 mg/day, or 350 mg/day, or 375 mg/day, or 400 mg/day, or 425 mg/day, or 450 mg/day.

More preferably, said metformin is present in an amount providing an effective dose of about 50 mg/day.

In one embodiment, the subject is a mammal, preferably a human, and said therapeutically effective dose of metformin is a daily dose to be administered in one take or in one injection.

In an embodiment, said glibenclamide is present in an amount providing an effective dose of between 0.001 mg/day and 2 mg/day.

In an embodiment, said glibenclamide is present in an amount providing an effective dose of between 0.001 mg/day and 1 mg/day.

Preferably, said glibenclamide is present in an amount providing an effective dose of 0.001 mg/day, or 0.005 mg/day, or 0.010 mg/day, or 0.015 mg/day, or 0.020 mg/day, or 0.025 mg/day, or 0.030 mg/day, or 0.035 mg/day, or 0.030 mg/day, or 0.035 mg/day, or 0.040 mg/day, or 0.045 mg/day, or 0.050 mg/day, or 0.055 mg/day, or 0.060 mg/day, or 0.065 mg/day, or 0.070 mg/day, or 0.075 mg/day, or 0.080 mg/day, or 0.085 mg/day, or 0.090 mg/day, or 0.095 mg/day, or 0.1 mg/day, or 0.15 mg/day, or 0.2 mg/day, or 0.25 mg/day, or 0.3 mg/day, or 0.35 mg/day, or 0.4 mg/day, or 0.45 mg/day, 0.5 mg/day, or 0.55 mg/day, or 0.6 mg/day, or 0.65 mg/day, or 0.7 mg/day, or 0.75 mg/day, or 0.8 mg/day, or 0.85 mg/day, or 0.9 mg/day, or 0.95 mg/day, or 1 mg/day, or 1.05 mg/day or 1.1 mg/day, or 1.15 mg/day, or 1.2 mg/day, or 1.25 mg/day, or 1.3 mg/day, or 1.35 mg/day, or 1.4 mg/day, or 1.45 mg/day, or 1.5 mg/day, or 1.55 mg/day, or 1.6 mg/day, or 1.65 mg/day, or 1.7 mg/day, or 1.75 mg/day, or 1.8 mg/day, or 1.85 mg/day, or 1.9 mg/day, or 1.95 mg/day, or 2 mg/day.

In an embodiment, said glibenclamide is present in an amount providing an effective dose of 0.5 mg/day.

In one embodiment, the subject is a mammal, preferably a human, and said therapeutically effective dose of glibenclamide is a daily dose to be administered in one take or in one injection.

According to the present invention, metformin and glibenclamide in a combination of the invention are to be administered either simultaneously, separately or sequentially with respect to each other.

According to one embodiment, said method for treating Parkinson's disease in a subject in need thereof comprises administering to the subject a combination comprising or consisting of metformin and glibenclamide as described hereinabove, wherein a therapeutically effective dose of metformin is administered once to the subject; then a therapeutically effective dose of glibenclamide is administered to the subject, preferably at least 1, 2, 3, 4, 5, 6, 7, 8, 9, or 10 days after the administration of a therapeutically effective dose of metformin.

According to one embodiment, said method for treating Parkinson's disease in a subject in need thereof comprises administering to the subject a combination comprising or consisting of metformin and glibenclamide as described hereinabove, wherein a therapeutically effective dose of glibenclamide is administered once to the subject; then a therapeutically effective dose of metformin is administered to the subject, preferably at least 1, 2, 3, 4, 5, 6, 7, 8, 9, or 10 days after the administration of a therapeutically effective dose of metformin.

In an embodiment, the pharmaceutical composition according to the invention further comprises at least one other acceptable active pharmaceutical ingredient, and/or at least one acceptable pharmaceutical excipient or carrier.

In a preferred embodiment, said active pharmaceutical ingredient is another anti-Parkinson's disease agent.

In a more preferred embodiment, said anti-Parkinson's disease agent is selected from the group consisting of L-DOPA, dopamine agonists, MAO inhibitors, COMT inhibitors, amantadine, carbidopa-levodopa, dopamine agonists, istradefylline, inosine, isradipine, apomorphine, donepezil, varenicline, foslevodopa-foscarbidopa and anti-cholinergics.

Levodopa (or L-dopa) is converted to dopamine in the brain. It is the reference treatment for Parkinson's disease, with the best efficacy/side-effect ratio. To prolong its effect, all the drugs in which it is found also contain a substance that inhibits its breakdown by the body.

Dopamine agonists are used in patients with Parkinson disease. Dopamine agonists, such as ropinirole, are the first-line treatment for restless legs syndrome, while bromocriptine is given for neuroleptic malignant syndrome. Dopamine agonists are also prescribed to counteract dopamine antagonist-induced hyperprolactinemia.

MAO-B inhibitor makes more dopamine available to the brain. They may be useful as early monotherapy (a medication used alone) or as an add-on to other medications, including levodopa. When used with other medications, MAO-B inhibitors may reduce motor fluctuations—periods of diminished symptom control as a levodopa dose wanes—minimizing “off” time and extending “on” time.

Amantadine (brand names Gocovri©, Symadine©, and Symmetrel©) is a medication used to treat dyskinesia associated with parkinsonism. Amantadine is the organic compound 1-adamantylamine or 1-aminoadamantane, which consists of an adamantane backbone with an amino group substituted at one of the four tertiary carbons. Rimantadine is a closely related adamantane derivative with similar biological properties

Carbidopa/levodopa, also known as levocarb and co-careldopa, is the combination of the two medications carbidopa and levodopa. It is primarily used to manage the symptoms of Parkinson's disease, but it does not slow down the disease or stop it from getting worse. It is taken by mouthdopamine agonists,

Foslevodopa-foscarbidopa is a soluble formulation of levodopa/carbidopa with solubility that allows for subcutaneous infusion for the treatment of motor complications for patients with Parkinson's disease.

Istradefylline, sold under the brand name Nourianz, is a medication used as an add-on treatment to levodopa/carbidopa in adults with Parkinson's disease (PD) experiencing “off” episodes. Istradefylline reduces “off” periods resulting from long-term treatment with the antiparkinson drug levodopa. An “off” episode is a time when a patient's medications are not working well, causing an increase in PD symptoms, such as tremor and difficulty walking.

Inosine is a nucleoside that is formed when hypoxanthine is attached to a ribose ring (also known as a ribofuranose) via a β-N9-glycosidic bond. Earlier trials suggested that patients with the highest serum urate levels had slower progression of Parkinson's symptoms. The trial uses inosine to raise urate levels in those with levels lower than the population mean (6 mg/dL).

Isradipine (tradenames DynaCirc, Prescal) is a calcium channel blocker of the dihydropyridine class.

Apomorphine, sold under the brand name Apokyn among others, is a type of aporphine having activity as a non-selective dopamine agonist which activates both D2-like and, to a much lesser extent, D1-like receptors. It also acts as an antagonist of 5-HT2 and α-adrenergic receptors with high affinity. Apomorphine is used in advanced Parkinson's disease intermittent hypomobility (“off” episodes), where a decreased response to an anti-Parkinson drug such as L-DOPA causes muscle stiffness and loss of muscle control.

Donepezil, sold under the brand name Aricept among others, is a medication used to treat dementia associated with Parkinson disease: Some evidence suggests that donepezil can improve cognition, executive function, and global status in Parkinson disease dementia.

Varenicline sold under the brand names Chantix and Champix among others, is a nicotinic receptor partial agonist and a cholinergic agonist. This molecule is suspected to have effect on balance and cognition in patients with Parkinson's disease.

Anticholinergics (anticholinergic agents) are substances that block the action of the neurotransmitter called acetylcholine (ACh) at synapses in the central and peripheral nervous system. These agents inhibit the parasympathetic nervous system by selectively blocking the binding of ACh to its receptor in nerve cells. The nerve fibers of the parasympathetic system are responsible for the involuntary movement of smooth muscles present in the gastrointestinal tract, urinary tract, lungs, sweat glands, and many other parts of the body. In broad terms, anticholinergics are divided into two categories in accordance with their specific targets in the central and peripheral nervous system and at the neuromuscular junction: antimuscarinic agents, and antinicotinic agents (ganglionic blockers, neuromuscular blockers).

In another embodiment, said active pharmaceutical ingredient is an antidiabetic agent, preferably chosen among: Repaglinide, Gliclazide, Glimepiride, Glipizide.

According to one embodiment, metformin and glibenclamide, the combination or pharmaceutical combination thereof, medicament or kit-of-parts according to the invention will be formulated for administration to the subject.

In an embodiment, the pharmaceutical composition is formulated to be suitable for oral, rectal, topical, intramuscular or intravenous or subcutaneous administration of the compounds.

In a preferred embodiment, the pharmaceutical composition is formulated to be suitable for oral administration.

In an embodiment, the oral administration is made by gelatin capsules, capsules, tablets, powders, granules, oral solutions or suspensions

In one embodiment, metformin as described hereinabove is in an adapted form for an oral administration. In another embodiment, metformin as described hereinabove is in an adapted form for an injection. Thus, in one, metformin as described hereinabove is to be injected to the subject, by intravenous, intramuscular, intraperitoneal, intrapleural, subcutaneous, transdermal injection or infusion. In one embodiment, glibenclamide as described hereinabove is in an adapted form for an oral administration.

Thus, in one embodiment, glibenclamide as described hereinabove is to be administered orally to the subject, for example as a capsule or as a tablet. In another embodiment, glibenclamide as described hereinabove is in an adapted form for an injection. Thus, in another embodiment, glibenclamide as described hereinabove is to be injected to the subject, by intravenous, intramuscular, intraperitoneal, intrapleural, subcutaneous, transdermal injection or infusion, preferably by intravenous injection.

In one embodiment, the combination, pharmaceutical combination, medicament or kit-of-parts according to the invention is in a form adapted for oral administration. In other words, the combination, pharmaceutical combination, medicament or kit-of-parts according to the invention comprises metformin and glibenclamide which are both in a form adapted for oral administration. Thus, in one embodiment, the combination, pharmaceutical combination, medicament or kit-of-parts according to the invention comprises metformin and glibenclamide which are to be administered orally to the subject.

Examples of forms adapted for oral administration include, without being limited to, liquid, paste or solid compositions, and more particularly tablets, tablets formulated for extended or sustained release, capsules, pills, dragees, liquids, gels, syrups, slurries, suspensions, and the like. In one embodiment, the combination, pharmaceutical combination, medicament or kit-of-parts according to the invention is in a form adapted for parenteral administration. In other words, the combination, pharmaceutical combination, medicament or kit-of-parts according to the invention comprises metformin and glibenclamide which are both in a form adapted for parenteral administration. Thus, in one embodiment, the combination, pharmaceutical combination, medicament or kit-of-parts according to the invention comprises metformin and glibenclamide which are to be administered parenterally.

In one embodiment, the combination, pharmaceutical combination, medicament or kit-of-parts according to the invention is in a form adapted for injection, such as, for example, for intravenous, subcutaneous, intramuscular, intradermal, transdermal injection or infusion. In other words, the combination, pharmaceutical combination, medicament or kit-of-parts according to the invention comprises metformin and glibenclamide which are both in a form adapted for injection, such as, for example, for intravenous, intramuscular, intraperitoneal, intrapleural, subcutaneous, transdermal injection or infusion. Thus, in one embodiment, the combination, pharmaceutical combination, medicament or kit-of-parts according to the invention comprises metformin and glibenclamide which are to be administered by injection to the subject, such as, for example, by intravenous, intramuscular, intraperitoneal, intrapleural, subcutaneous, transdermal injection or infusion.

Sterile injectable forms of metformin and glibenclamide, the combination or pharmaceutical combination thereof, or medicament according to the invention may be a solution or an aqueous or oleaginous suspension. These suspensions may be formulated according to techniques known in the art using suitable dispersing or wetting agents and suspending agents. The sterile injectable preparation may also be a sterile injectable solution or suspension in a non-toxic pharmaceutically acceptable diluent or solvent. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. In one embodiment, the combination, pharmaceutical combination, medicament or kit-of-parts according to the invention comprises metformin that is in a form adapted for oral administration and glibenclamide that is in a form adapted for injection, such as, for example, for intravenous, intramuscular, intraperitoneal, intrapleural, subcutaneous, transdermal injection or infusion. Thus, in one embodiment, the combination, pharmaceutical combination, medicament or kit-of-parts according to the invention comprises metformin that is to be administered orally and glibenclamide that is to be administered by injection to the subject, such as, for example, by intravenous, intramuscular, intraperitoneal, intrapleural, subcutaneous, transdermal injection or infusion. In one embodiment, the combination, pharmaceutical combination, medicament or kit-of-parts according to the invention comprises metformin that is in a form adapted for injection, such as, for example, for intravenous, intramuscular, intraperitoneal, intrapleural, subcutaneous, transdermal injection or infusion and glibenclamide that is in a form adapted for oral administration. Thus, in one embodiment, the combination, pharmaceutical combination, medicament or kit-of-parts according to the invention comprises metformin that is to be administered by injection to the subject, such as, for example, by intravenous, intramuscular, intraperitoneal, intrapleural, subcutaneous, transdermal injection or infusion and glibenclamide that is to be administered orally.

In another embodiment, the combination, pharmaceutical combination, medicament or kit-of-parts according to the invention is in a form adapted for topical administration. In other words, the combination, pharmaceutical combination, medicament or kit-of-parts according to the invention comprises metformin and glibenclamide which are both in a form adapted for topical administration. Thus, in one embodiment, the combination, pharmaceutical combination, medicament or kit-of-parts according to the invention comprises metformin and glibenclamide which are to be administered topically to the subject.

Examples of forms adapted for topical administration include, without being limited to, liquid, paste or solid compositions, and more particularly aqueous solutions, drops, dispersions, sprays, microcapsules, micro- or nanoparticles, polymeric patch, or controlled-release patch, and the like.

In another embodiment, the combination, pharmaceutical combination, medicament or kit-of-parts according to the invention is in a form adapted for rectal administration. In other words, the combination, pharmaceutical combination, medicament or kit-of-parts according to the invention comprises metformin and glibenclamide which are both in a form adapted for rectal administration. Thus, in one embodiment, the combination, pharmaceutical combination, medicament or kit-of-parts according to the invention comprises metformin and glibenclamide which are to be administered rectally.

Examples of forms adapted for rectal administration include, without being limited to, suppository, micro enemas, enemas, gel, rectal foam, cream, ointment, and the like.

In one embodiment, metformin is to be administered after glibenclamide. In one embodiment, metformin is to be administered at least 1, 2, 3, 4, 5, 6, 7, 8, or 10 days after glibenclamide.

In the description and in the following examples, unless otherwise indicated, ranges of values denominated as “between . . . and . . . ” include the lower and upper limits specified.

### BRIEF DESCRIPTION OF DRAWINGS

FIG. 1: Histogram presenting the impact of metformin (CXS001) and glibenclamide (CXS002) on dopaminergic neurons survival after 6-OHDA injury. It is shown that metformin (CXS001) and glibenclamide (CXS002) alone did not improve dopaminergic neuron survival in 6OHDA intoxication as compared to Brain Derived Neurotrophic Factor. CXS002 at high concentration seems to decrease neuron viability.

FIG. 2: Histogram presenting the impact of metformin (CXS001) and glibenclamide (CXS002) on neurites length after 6-OHDA injury. It is shown that metformin (CXS001) and glibenclamide (CXS002) alone did not improve dopaminergic neuron fitness (neurite length) in 6OHDA intoxication, as compared to Brain Derived Neurotrophic Factor.

FIG. 3: Histogram presenting the impact of metformin (CXS001) and glibenclamide (CXS002) on dopaminergic neurons survival after 6-OHDA injury. It is shown that metformin (CXS001) and glibenclamide (CXS002) at low dose improve dopaminergic neuron viability in 6OHDA intoxication. A tendency might be seen for metformin 10 μM+glibenclamide 10 μM, despite not being statistically significant at this dose.

FIG. 4: Histogram presenting the impact of metformin (CXS001) and glibenclamide (CXS002) on neurites length after 6-OHDA injury. It is shown that metformin (CXS001) and glibenclamide (CXS002) at low dose improves dopaminergic neuron fitness, estimated by mean neurite length, in 6OHDA intoxication.

FIG. 5: Representative pictures of the primary neuron cell cultures in standard conditions (A) or upon 6OHDA intoxication (B, C, D). Arrows point to viable dopaminergic neurons, differentially stained (dependent on tyrosine hydroxylase expression). Four conditions are represented: standard conditions (A); intoxication with 6OHDA (B); 6OHDA intoxication after 48-hour-pretreatment with metformin and glibenclamide (C); and 6OHDA intoxication after 48-hour-brain derived neurotrophic factor pretreatment (D).

FIG. 6: Effect of metformin (CXS001) and glibenclamide (CXS002) after 6-OHDA injury on survival of rat primary dopaminergic neurons expressed in percentage of control. Protective protocol (mean±s.e.m; * p<0.05, **** p<0.0001, stats vs 6-OHDA 20 μM; one-way ANOVA followed by Dunnett's test).

FIG. 7: Effect of metformin (CXS001) and glibenclamide (CXS002) in combination after 6-OHDA injury on survival of rat primary dopaminergic neurons expressed in percentage of control. Protective protocol (mean±s.e.m; * p<0.05, **** p<0.0001, stats vs 6-OHDA 20 μM; one-way ANOVA followed by Dunnett's test).

FIG. 8: Effect of metformin (CXS001) and glibenclamide (CXS002) in combination after 6-OHDA injury on survival of rat primary dopaminergic neurons expressed in percentage of control. Protective protocol (mean±s.e.m; * p<0.05, *** p<0.001, stats vs 6-OHDA 20 μM; one-way ANOVA followed by Dunnett's test).

FIG. 9: Effect of metformin (CXS001) and glibenclamide (CXS002) after 6-OHDA injury on neurites length of rat primary dopaminergic neurons expressed in percentage of control. Protective protocol (mean±s.e.m; * p<0.05, *** p<0.001, stats vs 6-OHDA 20 μM; one-way ANOVA followed by Dunnett's test).

FIG. 10: Effect of metformin (CXS001) and glibenclamide (CXS002) in combination after 6-OHDA injury on neurites length of rat primary dopaminergic neurons expressed in percentage of control. Protective protocol (mean±s.e.m; * p<0.05, **** p<0.0001, stats vs 6-OHDA 20 μM; one-way ANOVA followed by Dunnett's test).

FIG. 11: Effect of metformin (CXS001) and glibenclamide (CXS002) in combination after 6-OHDA injury on neurites length of rat primary dopaminergic neurons expressed in percentage of control. Protective protocol (mean±s.e.m; * p<0.05, *** p<0.001, stats vs 6-OHDA 20 μM; one-way ANOVA followed by Dunnett's test)

### EXAMPLES

### Example 1: Study of Potential Neuroprotector Effect of 2 Test Compounds and a Mix of these 2 Compounds after 6OH-DA Injury on Rat Primary Dopaminergic Neurons Survival: Analysis of the Number of Dopaminergic Neurons

6-hydroxydopamine (6OH-DA) is a selective catecholaminergic neurotoxin that is not only used as a pharmacological agent able to trigger PD-like stigmata (Sauer and Ortel, 1994; Cass et al., 2002) but also likely corresponds to a natural dopaminergic catabolite that accumulates in Parkinson's disease-affected brains and that appears to strongly contribute to this pathology (Jellinger et al., 1995). For this reason, 6OH-DA-induced dopaminergic neurotoxicity in mice is widely used as a model for Parkinson's disease research. Moreover, 6OH-DA inducing neurodegeneration of dopaminergic neurons in vitro, provides a useful model of Parkinson's disease.

This study will investigate the effect of 2 compounds and a mix of these 2 compounds at 3 concentrations on rat primary mesencephalic culture injured by 6OH-DA, a Parkinson′ disease in vitro model. For the Parkinson's disease, total number of dopaminergic neuron survival. BDNF will be used as positive control in this study.

A first culture of dopaminergic neurons will be carried out to determine the toxic concentrations of the compounds alone (at 4 concentrations) and the mix of the compounds (at 12 concentrations) in order to determine the maximum non-toxic concentrations to be used for the rest of the study.

A second and third culture of dopaminergic neurons will be prepared to study the protection of neurons in the presence of compounds after intoxication with 6 OHDA.

### Experimental Protocol

### 1. Dopaminergic Neuron Cell Culture

Rat dopaminergic neurons will be cultured as described by Schinelli et al., 1988. Briefly pregnant female rats of 15 days gestation will be killed by cervical dislocation (Rats Wistar; Janvier) and the foetuses will be removed from the uterus. The embryonic midbrains will be removed and placed in ice-cold medium.

Only the ventral portions of the mesencephalic flexure will be used for the cell preparations as this is the region of the developing brain rich in dopaminergic neurons. The midbrains will be dissociated by trypsinisation. Dissociated cells will be re-suspended in a defined culture medium and will be seeded at a in 96 well-plates and will be cultured at 37° C. in a humidified air (95%)/CO2 (5%) atmosphere.

Half of the medium will be changed every 2 days with fresh medium. In these conditions, after 5 days of culture, astrocytes are present in the culture and release growth factor allowing neurons differentiation. Three to six percent, of the neuronal cell population, are dopaminergic neurons.

After 6 days of culture, the culture medium will be changed by culture medium without growth factor.

### 2. Cytotoxicity of Compound

A first culture of dopaminergic neurons will be prepared for cytotoxicity test. Briefly, on day 7 of culture, cells will be incubated with compounds at 4 concentrations and with the mix of these 2 compounds at 16 concentrations treated for 48 h.

The following conditions will be done:
Control medium (vehicle to be defined, 48 h).
Control medium+CX001 at 1 mM, 100 μM, 10 μM and 1 μM.
Control medium+CX002 at 100 μM, 10 μM, 1 μM and 0.1 μM.
Control medium+CX001 at 1 mM+CX002 at 100 μM, 10 μM, 1 μM and 0.1 μM.
Control medium+CX001 at 100 μM+CX002 at 100 μM, 10 μM, 1 μM and 0.1 μM.
Control medium+CX001 at 10 μM+CX002 at 100 μM, 10 μM, 1 μM and 0.1 μM.
Control medium+CX001 at 1 μM+CX002 at 100 μM, 10 μM, 1 μM and 0.1 μM.

One culture will be performed with six wells per condition and three 96-well plate.

### 3. End Point Evaluation: Measurement of Total Number of TH Positive Neurons.

After 2 days of incubation, cells will be fixed by a solution of 4% paraformaldehyde for 20 min at room temperature, the control conditions will be fixed as well following the same procedure. The cells will be then permeabilized and non-specific sites will be blocked with a solution of phosphate buffered saline containing (PBS) with saponin and FCS for 15 min at room temperature. Cells will be incubated with Mouse Monoclonal Anti-Tyrosine Hydroxylase antibody (anti-TH, Sigma) in PBS with saponin and FCS overnight at 4° C. This antibody will be revealed with an Alexa Fluor 488 goat anti-mouse IgG in PBS with 1% FCS, 0.1% saponin, for 1 h at room temperature. Nuclei of cells will be labelled by a fluorescent marker (Hoechst solution) in the same solution.

For each well of culture, 20 pictures per well will be taken using InCell Analyzer™ 2200 (GE Healthcare) with 20× magnification. All the images will be taken in the same conditions. Analysis of cell bodies of TH positive neurons will be performed using Developer software (GE healthcare). A total of 6 data per experimental condition will be provided.

All values will be expressed as mean±s.e.mean. Statistical analyses will be done on the different conditions (ANOVA followed by Dunnett's test).

The highest non-toxic concentrations of the mixes will be chosen by the sponsor for the following studies.

### 4. 6OH-DA Exposure and Drug Treatment: Protective Protocol

A second and third first culture of dopaminergic neurons will be prepared for the 6OHDA challenge. On day 7 of culture, cells will be pre-treated for 1 h with test compound or reference compound then intoxicated with 6-OHDA (20 μM) for 48 h.

The following conditions will be done:

The following conditions will be done:
Control medium (vehicle to be defined, 48 h).
Control medium+6OHDA (20 μM, 48 h).
Control medium+CX001 at C1, C2 and C3+6OHDA (20 μM, 48 h).
Control medium+CX002 at C1, C2 and C3+6OHDA (20 μM, 48 h).
Control medium+CX001 at C1+CX002 at C1, C2 and C3+6OHDA (20 μM, 48 h).
Control medium+CX001 at C2+CX002 at C1, C2 and C3+6OHDA (20 μM, 48 h).
Control medium+CX001 at C3+CX002 at C1, C2 and C3+6OHDA (20 μM, 48 h).
BDNF (50 ng/ml; as reference compound+6OHDA (20 μM, 48 h).

Two cultures will be performed with six wells per condition and two 96-well plates per culture.

5. End Point Evaluation: Measurement of Total Number of TH Positive Neurons after 6OH-DA Intoxication.

After 2 days of incubation, cells will be fixed by a solution of 4% paraformaldehyde for 20 min at room temperature, the control conditions will be fixed as well following the same procedure. The cells will be then permeabilized and non-specific sites will be blocked with a solution of phosphate buffered saline containing (PBS) with saponin and FCS for 15 min at room temperature. Cells will be incubated with Mouse Monoclonal Anti-Tyrosine Hydroxylase antibody (anti-TH, Sigma) in PBS with saponin and FCS overnight at 4° C. This antibody will be revealed with an Alexa Fluor 488 goat anti-mouse IgG in PBS with 1% FCS, 0.1% saponin, for 1 h at room temperature. Nuclei of cells will be labelled by a fluorescent marker (Hoechst solution) in the same solution.

For each well of culture, 20 pictures per well will be taken using InCell Analyzer™ 2200 (GE Healthcare) with 20× magnification. All the images will be taken in the same conditions. Analysis of cell bodies of TH positive neurons will be performed using Developer software (GE healthcare). A total of 6 data per experimental condition will be provided.

All values will be expressed as mean±s.e.mean. Statistical analyses will be done on the different conditions (ANOVA followed by Dunnett's test).

### Results

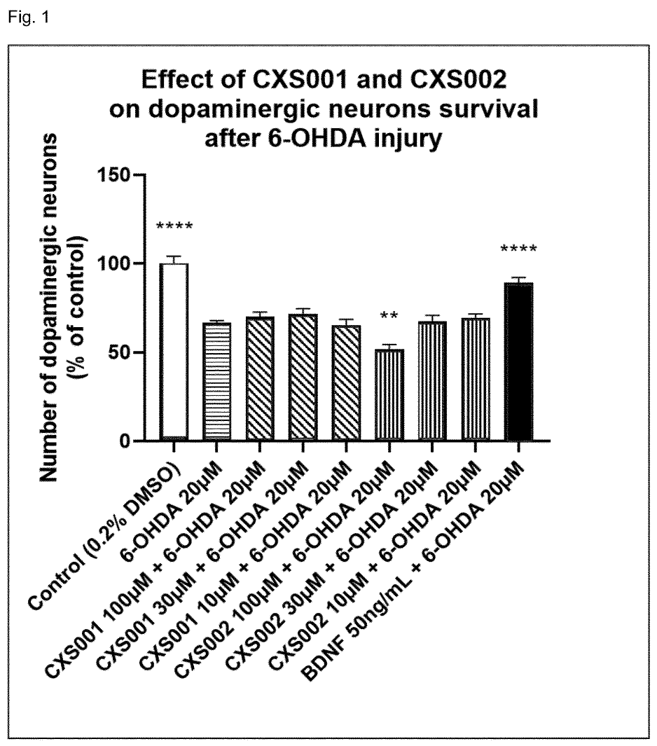

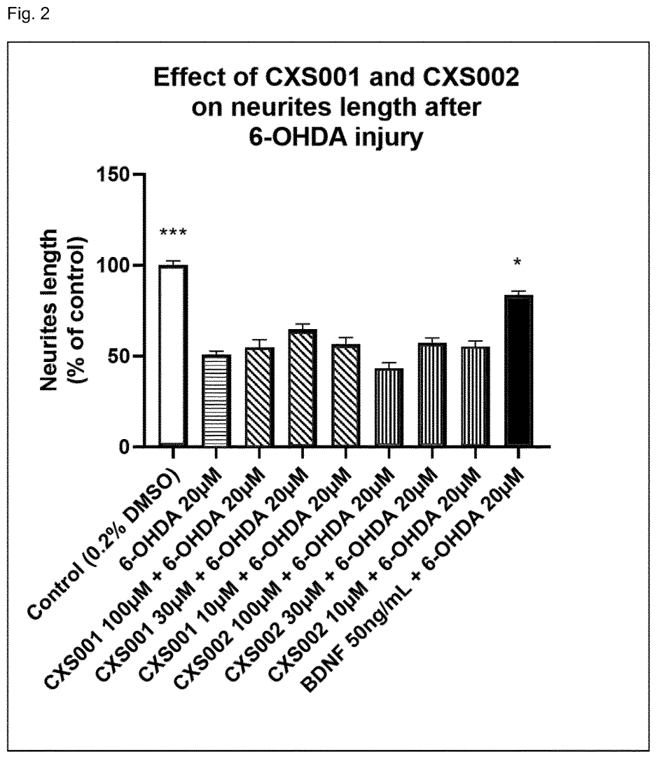

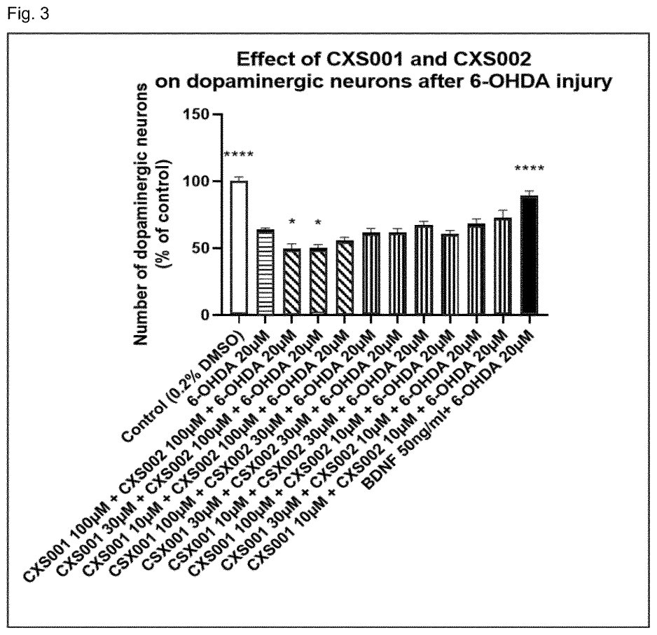

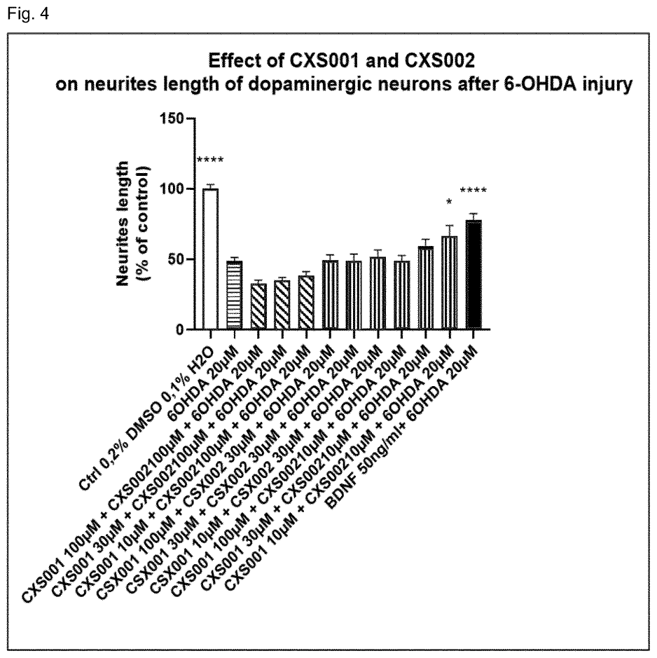

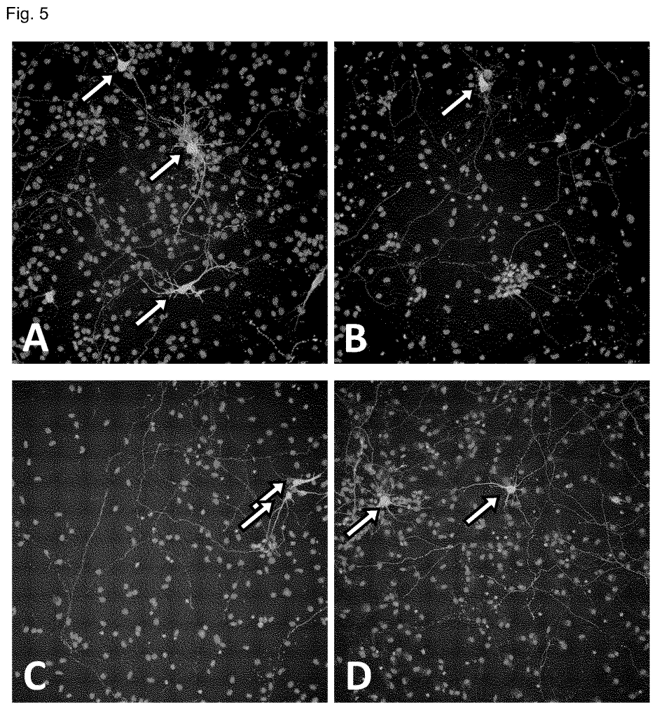

In vitro, metformin (CXS001) and glibenclamide (CXS002) were incubated both alone and in combination on primary cultures of rat neurons, containing approximately 5% of dopaminergic neurons (identified by tyrosine hydroxylase expression). The cultures, assessed for vitality (number of living dopaminergic neurons) and fitness (length of neurites) exhibited the results presented in FIGS. 1-5:
metformin (CXS001) and glibenclamide (CXS002) alone did not improve dopaminergic neuron survival in 6OHDA intoxication as compared to Brain Derived Neurotrophic Factor. CXS002 at high concentration seems to decrease neuron viability.
metformin (CXS001) and glibenclamide (CXS002) alone did not improve dopaminergic neuron fitness (neurite length) in 6OHDA intoxication, as compared to Brain Derived Neurotrophic Factor.
metformin (CXS001) and glibenclamide (CXS002) at low dose improve dopaminergic neuron viability in 6OHDA intoxication. A tendency might be seen for CXS001 10 μM+CXS002 10 μM, despite not being statistically significant at this dose.
metformin (CXS001) and glibenclamide (CXS002) at low dose improves dopaminergic neuron fitness, estimated by mean neurite length, in 6OHDA intoxication.

### Discussion

Metformin treatment has been associated with increased risk of developing Parkinson's disease in large studies, and glibenclamide is known to be potentially neurotoxic through excessive hypoglycemic effect. This example shows that a treatment involving low doses of metformin and glibenclamide can protect dopaminergic neurons against 6OHDA intoxication. Metformin reaches the brain, transported through the BBB by a transporter of the OCT family whilst glibenclamide remains outside, in healthy subjects. Yet, the BBB is subject to leakage in PD, this mechanism contributing to disease progression. By inhibiting the NLRP3/inflammasome complex on astrocytes, glibenclamide inhibit their activation in the brain compartment in case of BBB hyper permeability. By inhibiting the NLRP3/inflammasome on endothelial cells of the BBB themselves, glibenclamide strengthen it, thus preventing further leakage. This justifies discontinuous administration of glibenclamide in a combinatory treatment also involving metformin.

### Example 2: Study of Potential Neuroprotective Effect of the Combination According to the Invention Alone or in Combination after 6-OHDA Injury on Rat Primary Dopaminergic Neurons Survival. A Model of Parkinson's Disease

The aim of this study was to investigate the neuroprotective effect of metformin (CXS001) and glibenclamide (CXS002) alone or in combination, at 3 different concentrations, on survival of rat primary dopaminergic culture injured by 6-Hydroxydopamine (6-OHDA), a Parkinson's disease in vitro model. The total number and the neurites length of dopaminergic neurons was assessed. Brain Derived Neurotrophic factor (BDNF) was used has reference compound.

### Experimental Protocol

### Dopaminergic Neurons Cell Culture

Rat mesencephalic neurons were cultured as described by Schinelli et al., 1988. Briefly pregnant female rats of 15 days gestation were killed by cervical dislocation (Rats Wistar; Janvier Lab) and the fetuses removed from the uterus. The embryonic midbrains were removed and placed in ice-cold medium of Leibovitz 15 (L15; PanBiotech, Ref P04-27055, Batch: 1260323) containing 2% of Penicillin-Streptomycin (PS; PanBiotech, ref: P06-07100, Batch: 2585615) and 1% of bovine serum albumin (BSA; Sigma, Ref: 810533, Batch: 73). Only the ventral portions of the mesencephalic flexure were used for the cell preparation as this region of the developing brain is enriched in dopaminergic neurons. The midbrains were dissociated by 31rypsinization for 20 minutes (min) at 37° C. (Trypsin EDTA 1×; PanBiotech, Ref: P10-023100, Batch: 8290223). The reaction was stopped by the addition of Dulbecco's modified Eagles medium (DMEM; PanBiotech, Ref: P04-03600, Batch: 6041222) containing Dnase | grade II (0.1 mg/ml; PanBiotech, Ref: P60-37780100, Batch: H210916) and 10% of fetal calf serum (FCS; Invitrogen, Ref: 10270106, Batch: 2534381). Cells were then mechanically dissociated by 3 passages through a 10 mL pipette. Cells were then centrifuged at 180×g for 10 min at +4° C. on a layer of BSA (3.5%) in L15 medium. The supernatant was discarded and the cell pellets were resuspended in a defined culture medium consisting of Neurobasal Plus (Gibco, Ref: A3582901, Batch: 2537248) supplemented with 2% of B27 Plus (Gibco, ref: A3582801, Batch: 2722550), L-glutamine (2 mM; PanBiotech, Ref: P04-80100, Batch: 7061121), 2% of PS, 10 ng/ml of BDNF PeproTech, Ref: 450-02, Batch: 092361) and 1 ng/ml of Glial Derived Neurotrophic factor (GDNF; PeproTech, Ref: 450-10, Batch: 012364). Viable cells were then counted in a Neubauer cytometer using the trypan blue exclusion test. The cells were seeded at a density of 40 000 cells/well in 96 well-plates coated with poly-L-Ornithine (Sigma, Ref: P4957, Batch: RNBL6953) and laminin (Sigma, Ref: L2020, Batch: 0000216258), and were cultured at 37° C. in a humidified air (95%)/CO2 (5%) atmosphere. Half of the medium was changed every 2 days with fresh medium. In these conditions, after 5 days of culture, astrocytes are present in the culture and release growth factor allowing neurons differentiation. In this condition, 2 to 5% of neurons are dopaminergic neurons.

### 6-OHDA Preparation, Exposure and Drug Treatment Dopaminergic Neurons Cell Culture

Briefly, 6-OHDA (Sigma, Ref: H4381, Batch: MKCQ5002) was reconstituted in define culture medium at 40 μM (stock solution). The control medium was prepared in the same conditions. After 7 days of culture, primary mesencephalic neurons were pre-treated for 1 hour with test compounds or reference compound (BDNF, 50 ng/ml) and then intoxicated with 6-OHDA at a final concentration of 20 μM for 2 days incubation in order to induce a neuronal cell death of about 40%. The following conditions were done:

### Plate 1:

Control (0.05% DMSO)
Control+6-OHDA (20 μM, 2 days)
CXS001 at 10 μM, 3 μM and 1 μM+6-OHDA (20 μM, 2 days)
CXS002 at 10 μM, 3 μM and 1 μM+6-OHDA (20 μM, 2 days)
BDNF at 50 ng/ml+6-OHDA (20 μM, 2 days)

### Plate 2:

Control (0.05% DMSO)
Control+6-OHDA (20 μM, 2 days)
CXS001 at 10 μM+CXS002 at 10 μM, 3 μM and 1 μM+6-OHDA (20 μM, 2 days)
CXS001 at 3 μM+CXS002 at 10 μM, 3 μM and 1 μM+6-OHDA (20 μM, 2 days)
BDNF at 50 ng/ml+6-OHDA (20 μM, 2 days)

### Plate 3:

Control (0.05% DMSO)
Control+6-OHDA (20 μM, 2 days)
CXS001 at TUM+CXS002 at 10 μM, 3 μM and 1 μM+6-OHDA (20 μM, 2 days)
BDNF at 50 ng/ml+6-OHDA (20 μM, 2 days)

Two culture was performed with 6 wells per condition.

End point evaluation: measurement of total number of TH positive neurons and length of TH positive neurites

After 2 days of intoxication, cells were fixed by a solution of 4% paraformaldehyde (Alpha Aesar, ref J19943, Batch: 211457) for 20 min at room temperature, the control conditions were fixed as well following the same procedure. The cells were then permeabilized and non-specific sites were blocked with a solution of phosphate buffered saline (PBS; VWR; ref: L0615-500, Batch: MS01 MB) containing 0.1% of saponin (Sigma; ref: S7900, Batch: BCBL8667V) and 1% FCS for 15 min at room temperature. Cells were incubated with a rabbit polyclonal anti-Tyrosine Hydroxylase antibody (TH, 1/1000, Sigma, ref: AB152, Batch: 3870479) in a solution of PBS overnight at 4° C.

Staining was revealed with the addition of an Alexa Fluor 568 goat anti-rabbit IgG ( 1/400, Molecular probe, ref: A11011, Batch: 2500544) in PBS with 1% FCS and 0.1% saponin for 1 hour at room temperature. Nuclei of cells were labelled by a fluorescent marker (Hoechst, Sigma; ref: B1155, Batch: 046M4048V) in the same solution.

For each condition, 20 pictures per well were taken using InCell Analyzer™ 2200 (GE Healthcare) with 20× magnification. Images of each culture well were taken in same condition. The number of dopaminergic neurons (TH) and the neurite length of dopaminergic neurons are automatically evaluated with Developer system analysis (GE Healthcare). A total of 6 data per experimental condition were provided.

### Statistics

The data were expressed as mean±s.e.mean (of 6 data per condition, 1 culture). À global analysis of the data was performed using a one-way analysis of variance (ANOVA) following by Dunnett's test. The level of significance is set at p<0.05.

### Results

Effect of CXS001 and CXS002 on Dopaminergic Neurons Survival after 6-OHDA Injury (Plate 1)

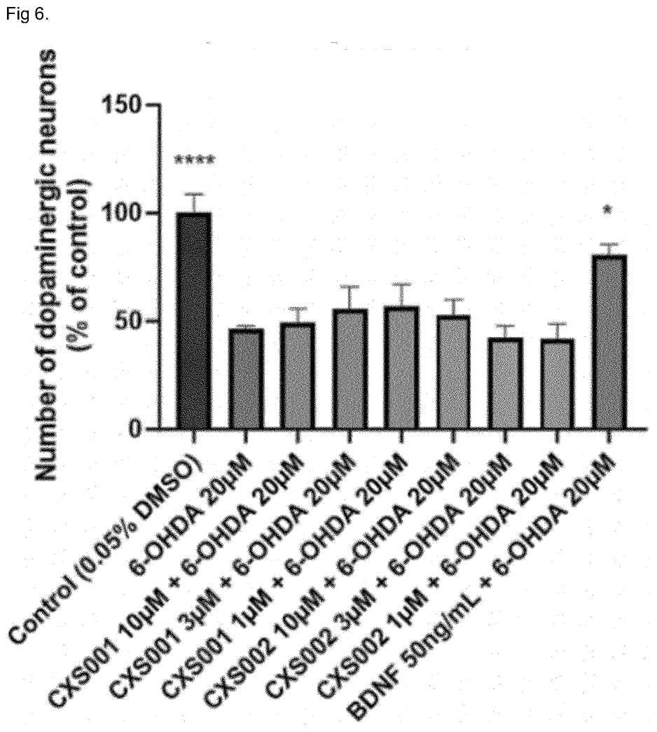

As observed on FIG. 6, a treatment with 6-OHDA at 20 μM during 48 hours induces a significant decrease of dopaminergic neurons survival (54% of cellular death, p<0.0001). As expected, the reference molecule, BDNF at 50 ng/ml, applied during 48 hours is able to significantly rescue neurons from cell death (81% of the control, p<0.05). These results allow validating the culture conditions.

A one-hour pre-treatment with CXS001 at 10 μM, 3M and 1 μM fails to significantly rescue dopaminergic neurons from cell death induced by 6-OHDA exposure during 48 hours (respectively 51%, 44% and 43% of cellular death).

Moreover, a one-hour pre-treatment with CXS002 at 10 μM, 3 μM and 1 μM fails to significantly rescue dopaminergic neurons from cell death induced by 6-OHDA exposure during 48 hours (respectively 47%, 57% and 58% % of cellular death).

Effect of CXS001 and CXS002 on Dopaminergic Neurons Survival after 6-OHDA Injury (Plate 2)

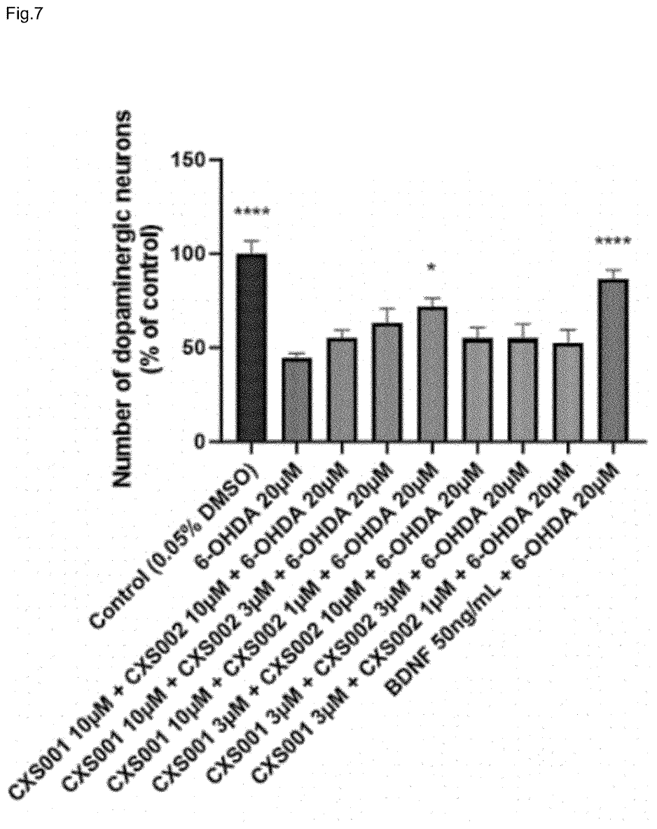

As observed on FIG. 7, a treatment with 5-OHDA at 20 μM during 48 hours induces a significant decrease of dopaminergic neurons survival (55% of cellular death, p<0.0001). As expected, the reference molecule, BDNF at 50 ng/ml, applied during 48 hours is able to significantly rescue neurons from cell death (87% of the control, p<0.0001). These results allow validating the culture conditions.

A one-hour pre-treatment with CXS001 at 10 μM in combination with CXS002 at 10 μM and 3 μM fails to significantly rescue dopaminergic neurons from cell death induced by 6-OHDA exposure during 48 hours (respectively 45% and 37% of cellular death). However, a co-treatment with CXS001 at 10 μM and CXS002 at 1 μM is able to significantly rescue dopaminergic neurons from cell death (72% of the control, p<0.05).

Moreover, λ one-hour pre-treatment with CXS001 at 3 μM in combination with CXS002 at 10 μM, 3M and 1 μM fails to significantly rescue dopaminergic neurons from cell death induced by 6-OHDA exposure during 48 hours (respectively 45%, 45% and 47% of cellular death).

Effect of CXS001 and CXS002 on Dopaminergic Neurons Survival after 6-OHDA Injury (Plate 3)

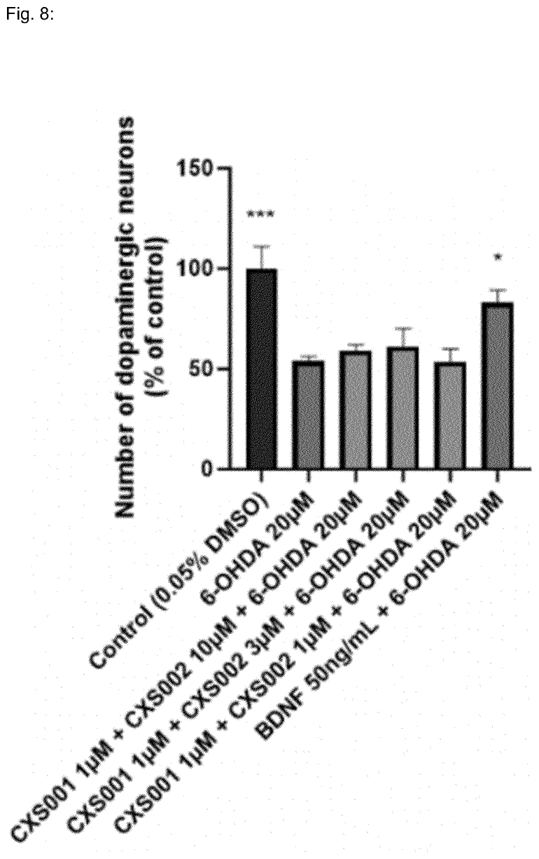

As observed on FIG. 8, a treatment with 6-OHDA at 20 μM during 48 hours induces a significant decrease of dopaminergic neurons survival (46% of cellular death, p<0.001). As expected, the reference molecule, BDNF at 50 ng/ml, applied during 48 hours is able to significantly rescue neurons from cell death (83% of the control, p<0.05). These results allow validating the culture conditions.

A one-hour pre-treatment with CXS001 at 1 μM in combination with CXS002 at 10 μM, 3 μM and 1 μM fails to significantly rescue dopaminergic neurons from cell death induced by 6-OHDA exposure during 48 hours (respectively 41%, 39% and 47% of cellular death).

Effect of CXS001 and CXS002 on Neurites Length of Dopaminergic Neurons after 6-OHDA Injury (Plate 1)

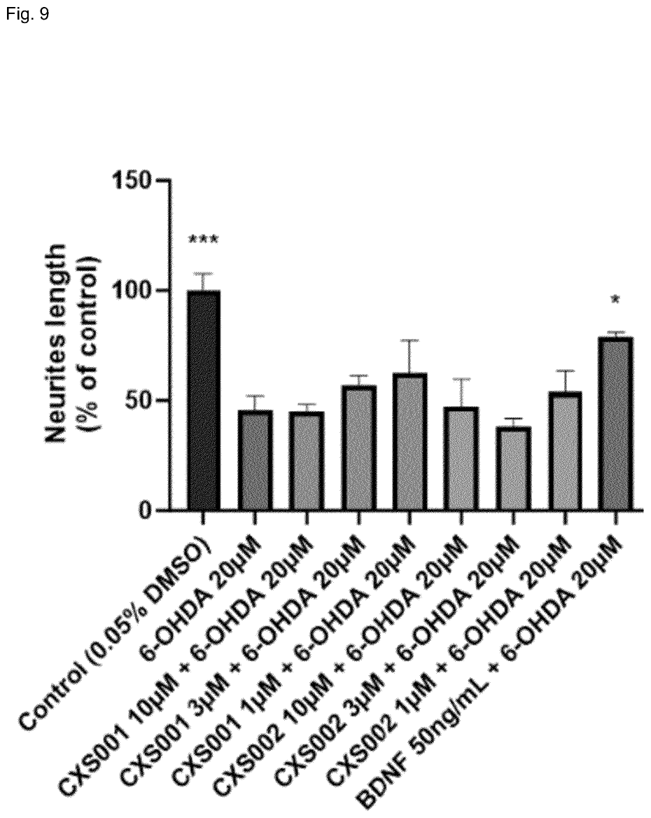

As observed on FIG. 9, a treatment with 6-OHDA at 20 μM during 48 hours induces a significant decrease of neurites length (54% of neurites loss, p<0.001). As expected, the reference molecule, BDNF at 50 ng/mL, applied during 48 hours is able to significantly rescue neurites length (79% of the control, p<0.05). These results allow validating the culture conditions.

A one-hour pre-treatment with CXS001 at 10 μM, 3 μM and 1 μM fails to significantly rescue neurites length of dopaminergic neurons after 6-OHDA exposure during 48 hours (respectively 55%, 43% and 38% of neurites loss).

Moreover, a one-hour pre-treatment with CXS002 at 10 μM, 3 μM and 1 μM fails to significantly rescue neurites length of dopaminergic neurons after 6-OHDA exposure during 48 hours (respectively 47%, 57% and 58% of neurites loss).

Effect of CXS001 and CXS002 on Neurites Length of Dopaminergic Neurons after 6-OHDA Injury (Plate 2)

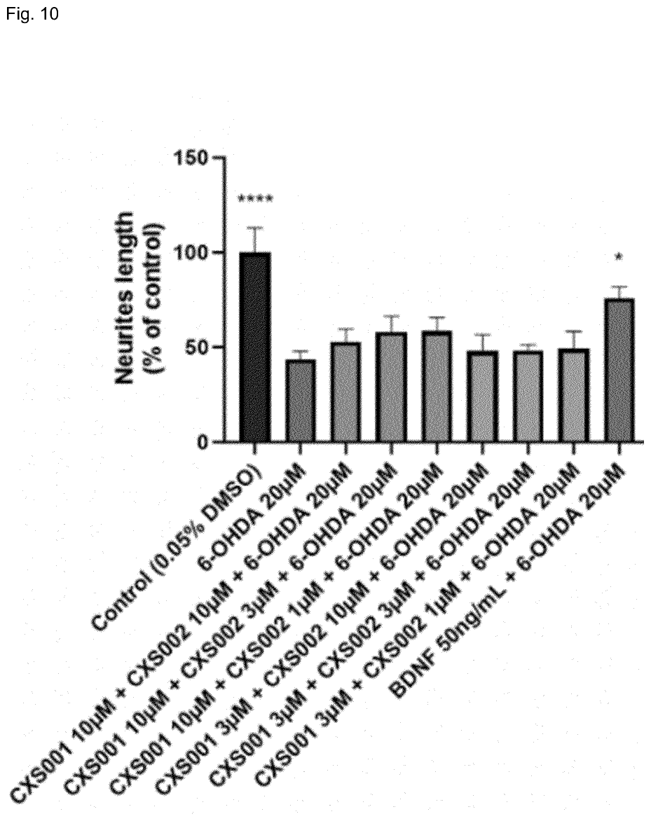

As observed on FIG. 10, a treatment with 6-OHDA at 20 μM during 48 hours induces a significant decrease of neurites length (56% of neurites loss, p<0.0001). As expected, the reference molecule, BDNF at 50 ng/ml, applied during 48 hours is able to significantly rescue neurites length (76% of the control, p<0.05). These results allow validating the culture conditions.

A one-hour pre-treatment with CXS001 at 10 μM in combination with CXS002 at 10 μM, 3 μM and 1 μM fails to significantly rescue neurites length of dopaminergic neurons after 5-OHDA exposure during 48 hours (respectively 47%, 42% and 42% of neurites loss).

Moreover, A one-hour pre-treatment with CXS001 at 3 μM in combination with CXS002 at 10M, 3 μM and 1 μM fails to significantly rescue neurites length of dopaminergic neurons after 6-OHDA exposure during 48 hours (respectively 52%, 52% and 41% of neurites loss).

Effect of CXS001 and CXS002 on Neurites Length of Dopaminergic Neurons after 6-OHDA Injury (Plate 3)

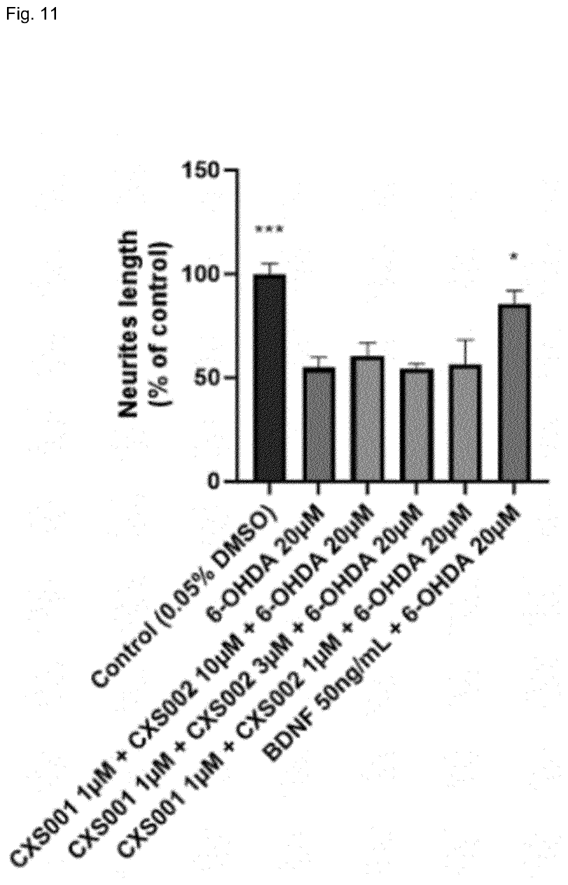

As observed on FIG. 11, a treatment with 6-OHDA at 20 μM during 48 hours induces a significant decrease of neurites length (45% of neurites loss, p<0.001). As expected, the reference molecule, BDNF at 50 ng/ml, applied during 48 hours is able to significantly rescue neurites length (85% of the control, p<0.05). These results allow validating the culture conditions.

A one-hour pre-treatment with CXS001 at 1 μM in combination with CXS002 at 10 μM, 3 μM and 1 μM fails to significantly rescue neurites length of dopaminergic neurons after 5-OHDA exposure during 48 hours (respectively 40%, 46% and 43% of cellular death).

### CONCLUSION

### Model Validation

As expected 6-OHDA applied at 20 μM during 2 days induces a significant loss of dopaminergic neurons and their neurites. Moreover, the reference molecule, BDNF at 50 ng/ml is able to significantly rescue these neurons from cell death.

### Metformin (CXS001) and Glibenclamide (CXS002)

A one-hour pre-treatment with CXS001 or CXS002 alone does not protect dopaminergic neurons from cell death and neurites from damages induced by 6-OHDA exposure during 48 hours.

A one-hour pre-treatment with CXS001 in combination with CXS002 does not significantly protect dopaminergic neurons from cell death or neurites from degradation induced by 6-OHDA exposure during 48 hours. However, the association of CXS001 at 10 μM with CXS002 at 1 μM partially and significantly protects dopaminergic neurons following 6-OHDA exposure during 48 hours.

1. A method of treating Parkinson's disease in a subject in need thereof, comprising administering metformin and glibenclamide.

2. The method of treating Parkinson's disease according to claim 1, wherein Parkinson's disease is early onset Parkinson's disease, idiopathic Parkinson's Disease, Parkinson's disease associated with aberrant splicing, trauma-induced Parkinson's disease, vascular Parkinson's disease, drug-induced or Parkinson's disease in the elderly adult.

3. The method of treating Parkinson's disease according to claim 1, wherein metformin is administered in an amount providing an effective dose of between 0.025 mg/day and 250 mg/day.

4. The method of treating Parkinson's disease according to claim 1, wherein metformin is administered in an amount providing an effective dose of about 50 mg/day.

5. The method of treating Parkinson's disease according to claim 1, wherein glibenclamide is administered in an amount providing an effective dose of between 0.001 mg/day and 2 mg/day.

6. The method of treating Parkinson's disease according to claim 1, wherein glibenclamide is administered in an amount providing an effective dose of 2 mg/day.

7. The method of treating Parkinson's disease according to claim 1, wherein glibenclamide is administered in an amount providing an effective dose of 0.5 mg/day.

8. The method of treating Parkinson's disease according to claim 1, wherein metformin and glibenclamide are administered concomitantly.

9. The method of treating Parkinson's disease according to claim 1, wherein metformin and glibenclamide are administered sequentially.

10. The method of treating Parkinson's disease according to claim 1, wherein the administration further comprises at least one additional active pharmaceutical ingredient and/or at least one pharmaceutically acceptable excipient or carrier.

11. The method of treating Parkinson's disease according to claim 10, wherein the additional active pharmaceutical ingredient is an anti-Parkinson's disease agent.

12. The method of treating Parkinson's disease according to claim 11, wherein the anti-Parkinson's disease agent is selected from the group consisting of L-DOPA, dopamine agonists, MAO inhibitors, COMT inhibitors, amantadine, carbidopa-levodopa, istradefylline, inosine, isradipine, apomorphine, donepezil, varenicline, foslevodopa-foscarbidopa, and anticholinergics.

13. The method of treating Parkinson's disease according to claim 1, wherein the administration is made through a pharmaceutical composition, formulated for oral, rectal, topical, intramuscular, intravenous, or subcutaneous administration.

14. The method of treating Parkinson's disease according to claim 13, wherein the pharmaceutical composition is formulated for oral administration.

15. The method of treating Parkinson's disease according to claim 14, wherein the oral administration is performed using gelatin capsules, capsules, tablets, powders, granules, oral solutions, or suspensions.
